(19) United States
(12) Patent Application Publication
(10) Pub. No.: US 2005/0101972 A1
(43) Pub. Date: May 12, 2005

(54) Devices and systems for separating and preparing skin

(75) Inventors: Nilkanth Bhalchandra Bhatavadekar, Chapel Hill, NC (US); David W. Mozingo, Gainesville, FL (US); Andrew Jordan Rapoff, Niskayuna, NY (US)

Correspondence Address: AKERMAN SENTERFITT, P.O. BOX 3188, WEST PALM BEACH, FL, 33402-3188, US

(73) Assignee: UNIVERSITY OF FLORIDA RESEARCH FOUNDATION, INC., GAINESVILLE, FL (US)

(21) Appl. No.: 10/962,839

(22) Filed: Oct. 12, 2004

Related U.S. Application Data
(60) Provisional application No. 60/517,996, filed on Nov. 6, 2003.

(51) Int. Cl. A61B 17/50; A61F 7/00

(52) U.S. Cl. 606/131

(57) ABSTRACT

A skin graft preparing system includes a surgical device having a housing with a front end defining a forward direction and a rear end defining a rear direction for moving along skin in the forward direction. Cutting structure is moveably coupled to the housing for separating skin from the body where the cutting structure has a cutting portion. The cutting portion cyclically translates in the forward and rear direction without lateral movement. The skin graft preparing system also includes a skin graft preparing device having a base structure, one or more skin feeding structures, one or more skin preparing structures, and one or more skin collecting structures coupled to the base structure. The skin supporting structure from the surgical device provides the skin feeding structure of the skin graft preparing device.

### CROSS-REFERENCE TO RELATED APPLICATIONS

This application claims priority to U.S. Provisional Patent Application 60/517,996 filed Nov. 6, 2003, and titled “Devices And Systems For Separating And Preparing Skin,” the entirety of which is herein incorporated by reference.

### FIELD OF THE INVENTION

The present invention relates to surgical systems, devices and methods, and more particularly, to skin-grafting systems, devices and methods.

### BACKGROUND OF THE INVENTION

Skin-grafting is a surgical procedure in which a piece of skin, the skin graft, is transplanted from one area of a body to another area of the body. In this surgical procedure, healthy or undamaged skin is separated from an area on a patient's body using a skin-cutting instrument, commonly referred to as a dermatome. The skin graft can be placed on the area of the patient's body to be covered, such as an area damaged by burns.

Devices for separating a skin graft for transplantation have included manually operated devices. Generally, these devices are used to manually excise a portion of skin for transplantation. Although a surgeon may carefully perform a skin separating procedure, manually operated skin separating devices can be susceptible to producing results that are affected by the surgeon. For example, during a surgical procedure of separating a portion of skin using a surgical device with a blade, the thickness of the separated skin can be determined by the motion of the blade. The thickness of the separated skin can further be determined by the pressure applied to the surgical device. Even surgeons who use extreme care while performing skin separating procedures can unintentionally apply variable pressure to the surgical device resulting in a skin graft of non-uniform thickness, jagged edges, and possibly irregular shape.

Furthermore, once a portion of skin is separated, the skin may be manipulated by another device to improve the usability of the skin in skin-grafting procedures. Devices that manipulate separated skin by providing many small incisions to allow the skin graft to be stretched to cover an area greater than the original size of the separated skin are commonly referred to as skin meshers. Although skin meshers can increase the area over which a skin graft can cover, non-uniform incision sizes and non-uniform arrangement of incisions can reduce the effectiveness of the skin graft. Furthermore, excessive skin manipulation by the operator using the skin mesher can lead to multiple disadvantages, such as the introduction of germs, non-uniform skin, and even irreparable damage to the skin.

### SUMMARY

The present invention provides a system and method for preparing skin grafts that can help to eliminate unsuccessful or rudimentary skin grafting procedures. The system includes a surgical device for separating skin without a lateral cutting motion that can result in separated skin with jagged edges. The system further includes a skin graft preparing device for preparing separated skin for a skin grafting procedure. Portions of the surgical device are interchangeable with the skin graft preparing system to limit manual manipulation of separated skin and introduction of possible infection. Thus, although the surgical device for separating skin and the skin graft preparing device can be used separately, when used together, they provide a complete system for separating skin and preparing skin grafts.

A surgical device for separating skin from a body includes a housing having a front end defining a forward direction and a rear end defining a rear direction for moving along skin in the forward direction. Cutting structure is moveably coupled to the housing for separating one or more portions of skin from the body where the cutting structure has a cutting portion. The cutting portion cyclically translates in the forward direction and the rear direction.

The housing can include a skin contacting surface and the cutting portion can translate substantially parallel to the skin contacting surface. The cutting portion can also translate at an angle to the skin contacting surface. The cutting portion can also translate a uniform forward distance, and therefore, the cutting structure can separate skin at a uniform depth. The housing can also include a guard to prevent unintentional skin separation.

The surgical device can also include a suction source for movably securing the housing to the skin. The suction source can be coupled to one or more suction ports located in the housing. The suction source can create a suction through the one or more suction ports to movably secure the housing to the skin. Additionally, a skin supporting structure that can be rotatably coupled to the housing can be included. The skin supporting structure can provide tension to and collect the separated skin. The skin supporting structure can be removably coupled to the housing and can include one or more attachment structures for attaching skin to the skin supporting structure. Also, the skin supporting structure can include a cylindrical body for receiving separated skin.

In another embodiment, a surgical device for separating skin from a body includes a housing and cutting structure movably coupled to the housing for separating one or more portions of skin from a body. The cutting structure can have an elongated cutting edge defining a first axis, and the cutting structure and the elongated cutting edge are substantially contained within a first plane. The elongated cutting edge cyclically translates in the first plane substantially perpendicular to the first axis.

The surgical device can also include a suction source for movably securing the housing to the skin. The suction source can be coupled to one or more suction ports located in the housing, where the suction source can create a suction through the one or more suction ports to movably secure the housing to the skin. A skin supporting structure can be rotatably coupled to the housing and can provide tension to and collect separated skin. The skin supporting structure can include one or more attachment structures for attaching skin to the skin supporting structure.

In accordance with the inventive arrangements, a skin graft preparing device can include a base structure, one or more skin feeding structures coupled to the base structure for feeding skin, one or more skin preparing structures coupled to the base structure for preparing the skin fed from the skin feeding structure, and one or more skin collecting structures coupled to the base structure for collecting the prepared skin from the skin preparing structure. The base structure can also include one or more first mounting structures and one or more second mounting structures for mounting the one or more skin feeding structures, one or more skin preparing structures, and one or more skin collecting structures. The first mounting structures and the second mounting structures can include a plurality of rotating elements for rotatably coupling one or more skin feeding structures, one or more skin preparing structures, and at least one skin collecting structures.

The rotating elements can be spatially arranged along the first mounting structure and the second mounting structure to allow for variable spatial coupling of the one or more skin feeding structures, the one or more skin preparing structures, and the one or more skin collecting structures. Additionally, the rotating elements can impart rotation motion to one or more of the one or more skin feeding structures, the one or more skin preparing structures, and the one or more skin collecting structures.

The one or more skin feeding structures, the one or more skin preparing structures, and the one or more skin collecting structures can be removably coupled to the base structure. Also, a dressing feeding structure can be rotatably and removably coupled to the base structure. The dressing feeding structure can be rotatably coupled to the base structure between the skin feeding structure and the skin preparing structure for coupling dressing to unprepared skin. The dressing feeding structure can also be rotatably coupled to the base structure between the skin preparing structure and the skin collecting structure for coupling dressing to prepared skin. The dressing feeding structure can be rotatably coupled to the base structure and can rotate in the clockwise direction so that the dressing from dressing feeding structure couples to a top side of the skin. Additionally, the dressing feeding structure can rotate in the counter-clockwise direction so that the dressing from dressing feeding structure couples to a bottom side of the skin.

Also in accordance with the inventive arrangements, a skin graft preparing system can include a surgical device having a housing with a front end defining a forward direction and a rear end defining a rear direction for moving along skin in the forward direction. A cutting structure can be included and can be moveably coupled to the housing for separating one or more portions of skin from the body. The cutting structure has a cutting portion which cyclically translates in the forward direction and the rear direction.

The skin graft preparing system can also include a skin graft preparing device having a base structure, one or more skin feeding structures rotatably and removably coupled to the base structure for feeding skin, one or more skin preparing structures rotatably coupled to the base structure for preparing the skin fed from the skin feeding structure, and one or more skin collecting structures rotatably coupled to the base structure for collecting the prepared skin from the skin preparing structure. The skin supporting structure from the surgical device provides the skin feeding structure of the skin graft preparing device. The skin graft preparing device can further include a dressing feeding structure rotatably and removably coupled to the base structure. Additionally, the skin graft preparing system can further include a skin applying device where the skin applying device rotatably couples to at least one of the skin supporting structure and the skin collecting structure.

A method of preparing a skin graft is provided and includes obtaining a portion of separated skin, preparing the portion of separated skin, and rotatably collecting the prepared portion of separated skin. The rotatably collecting step can include evenly distributing the prepared skin on a rotatable structure. The method can also include the step of feeding obtained separated skin for the preparing step. The preparing step can include one or more of cutting a plurality of incisions in the separated skin and placing a dressing in contact with the separated skin.

The preparing step can include first cutting a plurality of incisions in the separated skin and then placing a dressing in contact with the separated skin. The preparing step can also include first placing a dressing in contact with the separated skin and then cutting a plurality of incisions in one or more of the separated skin and the dressing. The dressing can be placed on a top surface of the separated skin and can be placed on a bottom surface of the separated skin.

Also in accordance with the inventive arrangements, a method of obtaining and applying a skin graft includes rotatably collecting separated skin and rotatably applying the separated skin. The skin can be rotatably collected and rotatably applied using the same rotatable structure.

### BRIEF DESCRIPTION OF THE DRAWINGS

There are shown in the drawings embodiments which are presently preferred, it being understood, however, that the invention is not limited to the precise arrangements and instrumentalities shown.

FIG. 1 is a perspective view of an embodiment of a surgical device for separating skin from a body.

FIG. 2A is a cross-sectional view of the surgical device for separating skin from a body in FIG. 1 taken along lines 2A-2A, with movement of the cutting structure shown in phantom.

FIG. 2B is a cross-sectional view of an alternative arrangement of the surgical device taken from a similar vantage point of the surgical device shown in FIG. 2A.

FIG. 3 is a cross-sectional view of the surgical device for separating skin in FIG. 1 taken along lines 3A-3A.

FIG. 4 is another view of the surgical device for separating skin in FIG. 1 showing a skin contacting surface of the housing.

FIG. 5 is a perspective view of one embodiment of a skin graft preparing device.

FIG. 6 is a perspective view of another embodiment of a skin graft preparing device.

FIG. 7 is a view of one embodiment of a skin graft preparing device showing the direction of rotation.

FIG. 8 is a view of another embodiment of a skin graft preparing device showing the direction of rotation.

FIG. 9 is a view of a device for applying the prepared skin to a patient.

FIG. 10 is a flow chart showing the steps of a method for preparing a skin graft.

FIG. 11 is a flow chart showing the steps of a method for obtaining and applying a skin graft.

### DETAILED DESCRIPTION OF THE PREFERRED EMBODIMENTS

The present invention provides systems, devices, and methods for preparing skin grafts that can help to eliminate unsuccessful or rudimentary skin grafting procedures. The system includes a surgical device for separating skin and a skin graft preparing device which can be used separately or in conjunction as a system. The system provides an integrated solution where components of the devices within the system are interchangeable during operation. Providing interchangeable components completely eliminates the transferring of separated skin from one device to the next, and therefore, greatly reduces chances of human error and the introduction of germs from unnecessary human handling of the separated skin. Although components of the surgical device and the skin graft preparing device are interchangeable and the devices complement each other, both devices can be used separately as they do not depend upon each other.

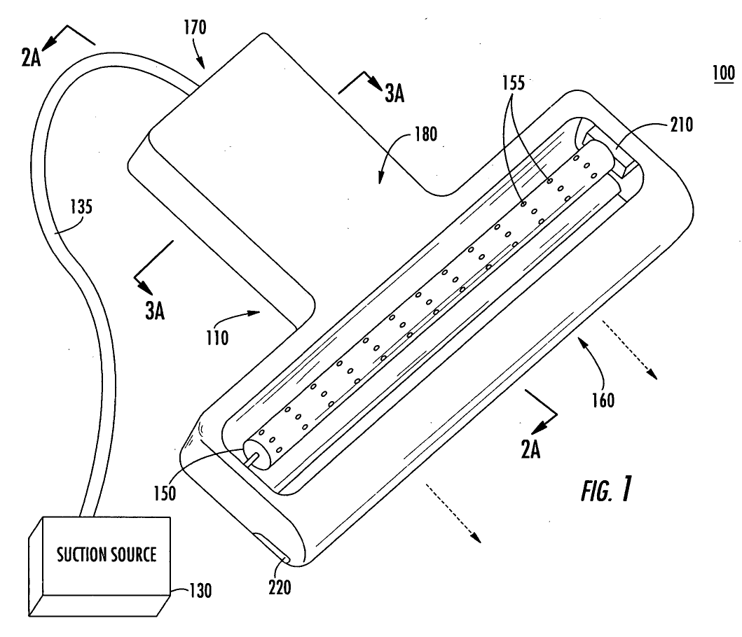

In accordance with the inventive arrangements, FIGS. 1 through 4 illustrate a variety of views of a surgical device 100 for separating skin and show the different components of the surgical device 100. The surgical device 100 includes a housing 110 adapted to be moved along the skin of a patient and a cutting structure 120 movably coupled to the housing 110. The cutting structure 120 can be used to separate one or more portions of the skin from a patient's body and can be adjusted by the cutting structure lever 115. The surgical device 100 can also include a suction source 130 coupled to one or more suction ports 140. Additionally, the surgical device 100 can include a skin supporting structure 150 that is rotatably coupled to the housing 110 and rotated by rotating mechanism 210.

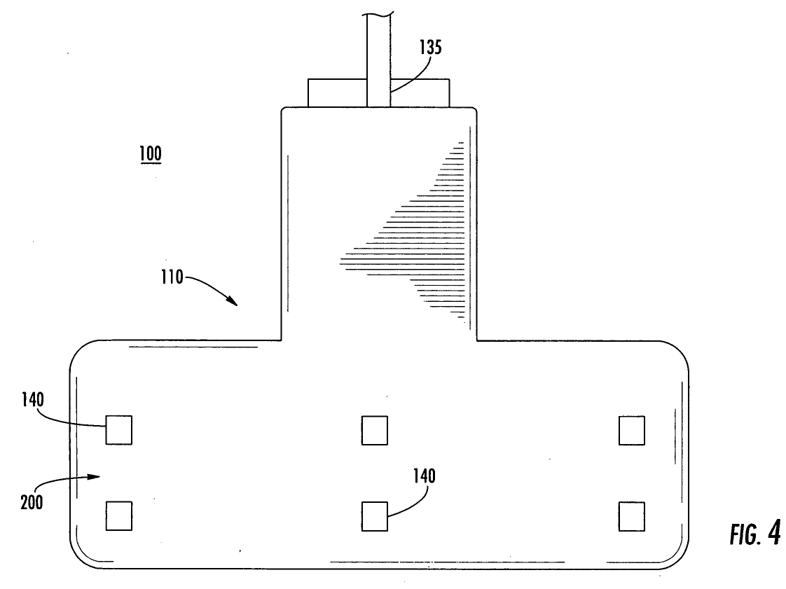

FIG. 1 is a perspective view of the surgical device 100 for separating skin that shows the housing 110 of one embodiment of the surgical device 100. The housing 110 houses the different components of surgical device 100, and therefore, can also be considered a housing of the surgical device 100. The housing 110 can be ergonomically shaped for ease of use. The housing 110 includes a front end 160 defining a forward direction, shown by arrows, and a rear end 170 defining a rear direction. The forward direction extends outward from the front end 160 of the housing 110 and is substantially parallel to the skin contacting surface 200 (shown in FIG. 4). Similarly, the rear direction extends outward from the rear end 170 of the housing 110 and is substantially parallel to the skin contacting surface 200 (shown in FIG. 4).

The housing 110 can include a handle portion 180 that is shaped and sized to be gripped by a user during operation of the surgical device 100. Generally, the housing 110 is adapted to move in the forward direction. Nevertheless, the invention is not limited in this regard as the housing 110 is not limited in the direction of motion along a patient's skin. For example, the components of surgical device 100 can be configured so that the housing 110 can be adapted to move in the rear direction while in operation.

Additionally, the housing 110 can include a guard 220 to prevent unintentional skin separation. The guard 220 forms a protective barrier between the cutting structure 120 and the patient's skin. Thus, the guard 220 prevents the cutting structure 120 from unintentional cutting when the surgical device 100 is not in use. During use, the guard 220 can be used to protect portions of the skin that are not intended to be separated.

The housing 110 can be constructed of any suitable material such as plastic, metal, and ceramic. Preferably, the housing 110 can be constructed any suitable material that can withstand medical industry standard serialization processes. A non-exhaustive list of such materials include surgical steels which do not degrade from exposure to high temperatures associated with sterilization by steam and plastics that do not degrade under high energy radiation sterilization. It should be noted that the invention is not limited in this regard as the housing 110 can be constructed of other materials that cannot withstand medical industry standard sterilization processes. Thus, the housing 110 can be designed for single patient use and can be considered as disposable.

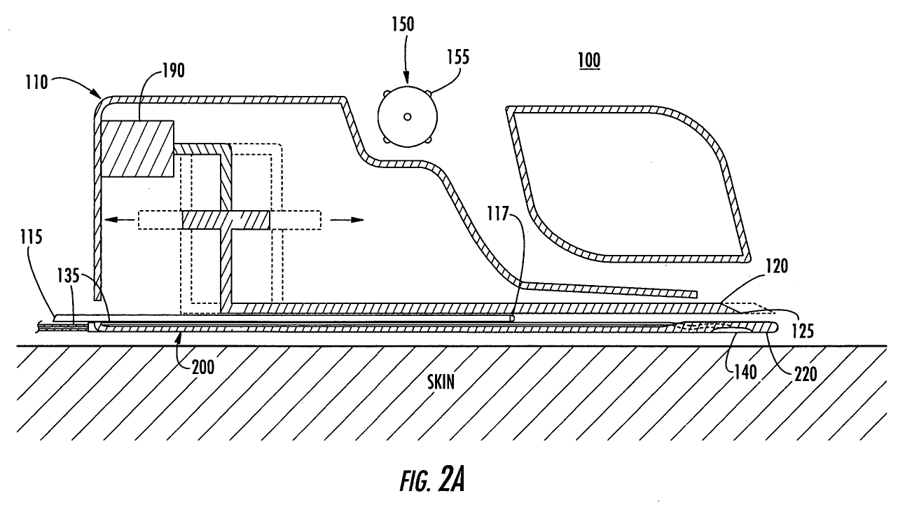

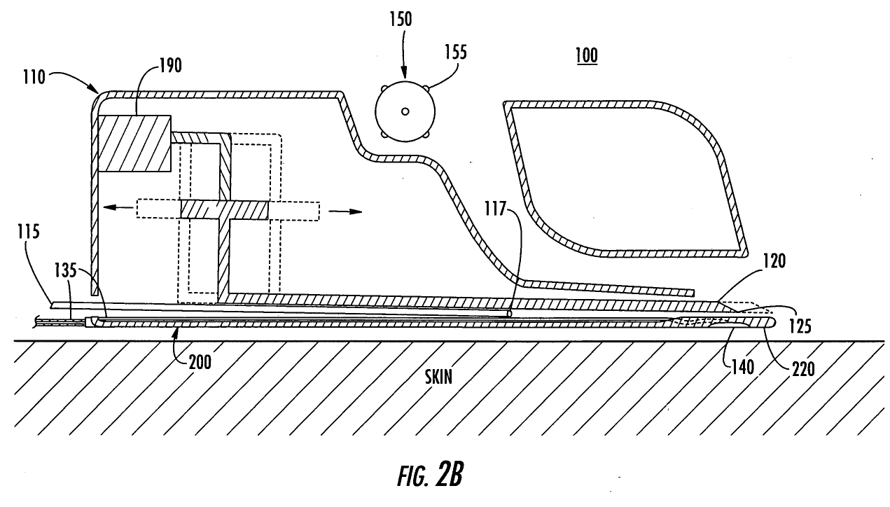

There is shown in FIG. 2A a cross-sectional view of the surgical device 100 taken along lines 2A-2A, resting on a patient's skin that has not been separated. The cutting structure 120 for separating at least a portion of the skin from a patient's body can be movably coupled to the housing 110. The cutting structure 120 has a cutting portion 125 that is used to separate the skin from the patient. The cutting structure 120 can include any suitable structure for separating skin from a patient's body. For example, the cutting structure 120 can include a typical blade with a sharpened straight edge as the cutting portion 125. Alternatively, the cutting structure 120 can include a blade with a serrated edge as the cutting portion 125. Additionally, the cutting structure 120 is not limited to a unitary structure and can include multiple components that can move relative to each other. Preferably, the cutting structure 120 is constructed of surgical steel; however, the invention is not limited in this regard as the cutting structure 120 can also be constructed of other suitable materials, such as steel alloys, ceramics, and the like. It should be noted that the cutting structure 120 can be reusable after undergoing medical industry standard sterilization processes and can also be disposable as being limited to use on one patient.

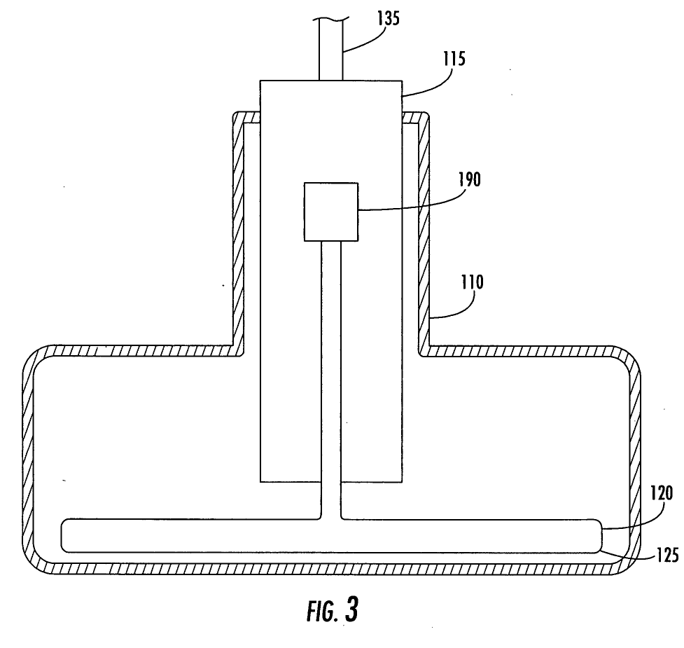

There is shown in FIG. 3, a cross-sectional view taken along line 3A-3A of the surgical device 100 shown in FIG. 1 is presented. This cross-sectional view shows the cutting structure 120 with cutting portion 125 as generally spanning the width of the front end 160 of the housing 110. In this embodiment, the cutting portion 125 provides a wide cutting surface for separating skin having a width generally matching the width of the housing 110. Nevertheless, the invention is not limited in this regard as the cutting structure 120 can be formed of any suitable shape and size, such as a relatively narrow cutting structure 120 for separating skin from narrow portions of a body.

The housing 110 includes a cutting structure actuator 190 that is coupled to the housing 110 (FIG. 2A). The cutting structure 120 can be coupled to the cutting structure actuator 190, which can cause at least the cutting portion 125 of cutting structure 120 to cyclically translate in the forward and rear direction. While the entire cutting structure 120 can translate in the forward and rear direction, the cutting structure 120 can include a component cutting structure 120 where only the cutting portion 125 cyclically translates in the forward and rear direction. Thus, the cutting structure 120 is movably coupled to the housing 110 as the cutting structure actuator 190 causes at least the cutting portion 125 to move relative to the housing 110.

The rate of cycles produced by the cutting structure actuator 190 can be defined by a movement of at least the cutting portion 125 translating from a retracted position to a cutting position and then returning to the retracted position. In most instances, a rate of approximately 10 to 25 cycles per second will be adequate for separating skin; however, some patients may require another cyclical rate. For instance, some patient's, such as different humans and different animals, may have very tough skin that is difficult to separate. Such patients may require a greater cyclical rate, such as 30 cycles/second, 35 cycles/second, 40 cycles/second, and so on, can be used to separate the skin. The cycle rate produced by the cutting structure actuator 190 can be set at a predetermined level before operation of the surgical device 100 and can be adjusted during operation to allow the user to react to varying skin conditions.

The cutting structure actuator 190 can include any suitable mechanism for cyclically translating at least the cutting portion 125. The cutting structure actuator 190 can include a cam, an electromagnetic motor, and the like. Nevertheless, the invention is not limited in this regard as one skilled in the art would readily understand the variety of cutting structure actuators 190 capable of producing movement of the cutting structure 120.

During the forward translation of the cutting portion 125, skin can be separated from a body. The cutting structure actuator 190 can be configured to translate the cutting portion 125 a uniform forward distance during each cycle. Thus, during the portion of the cycle in which skin is separated, the cutting portion 125 translates a uniform distance to ensure that skin is separated at a uniform depth. Additionally, the cutting structure actuator 190 causes at least the cutting portion 125 to translate only in the forward direction and rear direction. Translating in the forward and rear direction is contrasted to a lateral, or sawing movement, which can produce jagged and uneven edges of the portion of separated skin. Translating only in the forward and rear direction, without lateral movement, ensures that points at the edge of the intended area of skin to be separated are always in touch with cutting portion 125. Such forward translation without lateral movement produces separated skin with straight and even edges when compared to skin with jagged edges produced by a lateral cutting motion.

Separated skin with straight and even edges can easily be abutted against another portion of separated skin with straight and even edges to produce an even margin between the portions of separated skin. Adjacent portions of separated skin with uneven margins can produce pocking of the skin, leading to greater chance of infection. In addition to increased chance of infection, skin with jagged edges can produce undesirable aesthetic results which can be further highlighted when adjacent portions of skin have uneven margins.

Additionally, the cutting portion 125 of the cutting structure 120 can include an elongated cutting edge 125 shown in FIG. 3. The cutting structure 120 and the elongated cutting edge 125 can be substantially contained within a first plane that can extend outward from the surgical device 100 and is substantially parallel with skin to be separated. Additionally, the elongated cutting edge 125 defines a first axis that is also within the first plane. In operation, the elongated cutting edge cyclically may translate in the first plane substantially perpendicular to the first axis. Such a translation of the elongated cutting edge 125 perpendicular to the first axis can ensure that the edge of the intended area of skin to be separated will be a straight and even edge as the elongated cutting edge 125 does not move laterally.

As shown in FIG. 2A, the housing 110 can have a skin contacting surface 200 that rests against and contacts the skin of a patient. The cutting structure 120, particularly the cutting portion 125 is shown in phantom as translating substantially parallel to the skin contacting surface 200. In this arrangement, the cutting portion 125 is not angled into the skin, and therefore, the cutting portion 125 is prevented from advancing into the depths of the skin layers and also prevented from producing skin separation at non-uniform depths.

Nevertheless, the invention is not limited to at least a cutting portion 125 that translates substantially parallel with the skin contacting surface 200 of the housing 110. FIG. 2B illustrates another arrangement of the surgical device 100 shown in cross section where the cutting structure lever 115 is pivotally movable to angle the cutting structure 120. The cutting structure lever 115 can be pivotally coupled to the housing 110 at pivot 117. This arrangement allows the cutting structure lever 115 to be pivotally moved to position the cutting structure 120 at a variety of angles.

In this arrangement, instead of at least the cutting portion 125 translating substantially parallel with the skin contacting surface 200, the cutting portion 125 translates at an angle with the skin contacting surface 200. Although the cutting portion 125 is at an angle with the skin contacting surface 200, the cutting portion 125 still cyclically translates in the forward direction and the rear direction. Notably, the cutting structure lever 115 allows the angle at which the cutting structure 120 separates the skin to be altered during operation, which can be helpful when beginning and ending the skin separating process; nevertheless, the invention is not limited in this regard as the surgical device 100 can operate without a cutting structure lever 115. Additionally, the housing 110 can include a structure (not shown) for securing the cutting structure lever 115 at a particular angle. Markings (not shown) can also be provided on the housing 110 for displaying at which angle the cutting structure lever 115 is set.

Furthermore, although the cutting portion 125 may be angled down into the skin of a patient, the cutting structure actuator 190 causes the cutting portion 125 to translate a uniform distance in the forward direction. With the cutting portion 125 translating a uniform distance during each cycle, the cutting portion 125 can separate the skin at a uniform depth during each cycle. Such a uniform translation can ensure that the separated skin will be of uniform thickness resulting in usable skin grafts that can provide a better prognosis and aesthetic result when compared to skin grafts of non-uniform thickness of random shape and size and jagged margins.

The surgical device 100 can also include a suction source 130 (shown in FIG. 1) coupled to one or more suction ports 140 located in the housing 110. The suction source 130 can produce a vacuum pressure that has enough force to hold the surgical device 100 against the skin of a patient's body to ensure intimate contact and to withstand the reaction forces created by skin separation. Nevertheless, the suction source 130 should not produce a great enough vacuum pressure so that the surgical device 100 cannot be moved along the skin of the patient.

The suction source 130 can alleviate the operator of the surgical device 100 from supplying a continuous force to hold the surgical device 100 on the patient's body during operation. The suction source 130 also can produce a relatively constant pressure and can remove natural human error from the inability to produce such a constant pressure over time. Thus, the suction source 130 allows the user to operate the surgical device 100 without experiencing fatigue and introducing human error.

The vacuum pressure created by the suction source 130 can be varied according to the needs of the user and/or patient. For example, some patients can have tough skin and may require a greater vacuum pressure to secure the housing 110 to the skin as the force necessary to separate the skin is increased. Furthermore, the suction source 130 can be configured to allow for changes in vacuum pressure during operation of the surgical device 100. Nevertheless, it should be noted that the vacuum pressure created by the suction source 130 should not be increased to the level at which the housing 110 cannot be moved along the skin. Thus, the suction source 130 can provide an optimal vacuum pressure that can ensure intimate and uniform contact of the surgical device 100 wit the patient's skin while allowing the user to move the surgical device 100 along the patient's skin. The suction source 130 can include any source capable of creating a vacuum pressure at suction ports 140. For example, the suction source 130 can include a stand alone suction device and can also include a suction source, such as a typical connection provided in a health care office.

As shown in the figures, the suction source 130 can be connected to the suction ports 140 via tubing 135. The tubing 135 can be constructed of a flexible rubber material and the like. Tubing 135 can also be constructed of other materials. For instance, while the portions of the tubing 135 that are outside of the housing 110 can be a flexible rubber material, the portion of the tubing 135 within the housing 110 can be constructed of surgical steel or any other material capable of withstanding medical industry standard sterilization processes.

As shown in FIG. 4, the suction ports 140 can be located at various areas of the skin contacting surface 200. It should be noted that the invention is not limited to the arrangement of the suction ports 140 shown in FIG. 4. Other arrangements can include suction ports 140 that are aligned only along the lateral edges of the skin contacting surface 200 and a plurality of uniformly distributed suction ports 140 spanning over the entire skin contacting surface 200. Still other arrangements can include one suction port 140 that spans a relatively large region of the skin contacting surface 200.

Referring to FIGS. 1, 2A and 2B, the surgical device 100 can also include a skin supporting structure 150 that is rotatably coupled to the housing 110. Separated skin can be collected by progressively wrapping the separated skin around the skin supporting structure 150. Progressively wrapping the skin around the skin supporting structure 150 limits user manipulation of the skin and maintains the skin in a flat sheet that is optimal for future skin graft preparation. The skin supporting structure 150 is preferably cylindrical in shape and constructed of a material that can withstand medical industry standard sterilization processes. Nevertheless, the invention is not limited in this regard as the skin supporting structure 150 can include other shapes capable of being rotatably coupled to the housing 110 and can include other disposable materials.

The skin supporting structure 150 can be rotatably coupled to a rotating mechanism 210 that can rotate the skin supporting structure 150 as it collects separated skin. The skin supporting structure 150 and the rotating mechanism 210 combine to provide tension to partially separated skin. Further, it should be noted that the tension provided to the partially separated skin can ease the amount of vacuum pressure supplied by the suction source 130 as the tensed skin will combine with the vacuum pressure to ensure intimate contact of the surgical device 100.

The rotating mechanism 210 can be adjusted to provide optimal tension to ease the process of separating skin. Additionally, the rotating mechanism 210 can include a level of play, so that the rate of rotation is not always constant and can allow for slower uptake of portions of skin. Thus, the rotating mechanism 210 can be configured so as not to tear the skin if the skin is slowly collecting on the skin supporting structure 150. The rotating mechanism 210 can include any suitable mechanism for rotating the skin supporting structure 150 such as an electric motor.

The skin supporting structure 150 can also include one or more attachment structures 155 for attaching and securing skin to said skin supporting structure 150. The attachment structures can include small protrusions, such as spikes and/or ridges, which are able to securely attach a portion of separated skin to the supporting structure 150. Nevertheless, the attachment structures 155 are not limited to small protrusions and can include a layer of adhesive or other. As shown, the attachment structures 155 can be aligned in rows to intermittently attach to skin that is collected on the skin supporting structure 150.

In one embodiment, the skin supporting structure 150 can be removably coupled to the housing 110. A removably coupled skin support structure 150 can provide many benefits. In one instance, a removably coupled skin support structure 150 can be replaced with other skin supporting structures 150 and can also allow the skin supporting structure 150 to be individually sterilized with medical industry standard sterilization processes. As more fully discussed below, a removably coupled skin supporting structure 150 with collected separated skin can be incorporated in other devices for use.

In operation, the housing 110 can be placed on a patient's skin with the front end 160 orientated to travel forward over the region of skin to be separated. The suction source 130 can produce a vacuum pressure at the suction ports 140 to movably secure the housing 110 to the patient. The cutting structure actuator 190 can cause at least the cutting portion 125 of the cutting structure 120 to cyclically translate in the forward and rear directions and the rotating mechanism 210 can cause the skin supporting structure 150 to rotate. Lever 115 may be used to adjust the angle of the cutting structure 120.

The cutting portion 125 of the cutting structure 120 can separate a portions of the skin from the patient's body while the guard 220 protects the areas of skin that should not be separated. As the cutting portion 125 begins to separate skin from the patient, the skin can travel in the rear direction until a portion of the skin is engaged by the attachment structures 155 which can secure the separated portion of skin to the skin supporting structure 150. As the rotating mechanism 210 causes the skin supporting structure 150 to rotate, the separated skin will become progressively wrapped around the skin supporting structure 150.

It should be noted that as the skin supporting structure 150 collects separated skin, the rotating mechanism 210 provides tension to the skin by rotating the skin supporting structure 150. Nevertheless, the rotating mechanism 210 can rotate at various speeds, and therefore, the tension to the skin cannot be great enough to tear the skin as speed of the rotating mechanism can be decreased during operation. Accordingly, varying the rotation speed of skin supporting structure 150 can accommodate various skin types and prevent skin tearing. With the desired amount of skin separated from the body, the skin is completely severed from the patient's body and collected on the skin supporting structure 150. The collected skin can now be used for any suitable purpose, such as in a skin grafting procedure.

Also in accordance with the inventive arrangements, a skin graft preparing device is provided. A skin graft preparing device can manipulate and prepare the skin before a skin grafting procedure. Once the skin is prepared by the skin grafting structure, the skin can be collected for use during a skin graft procedure.

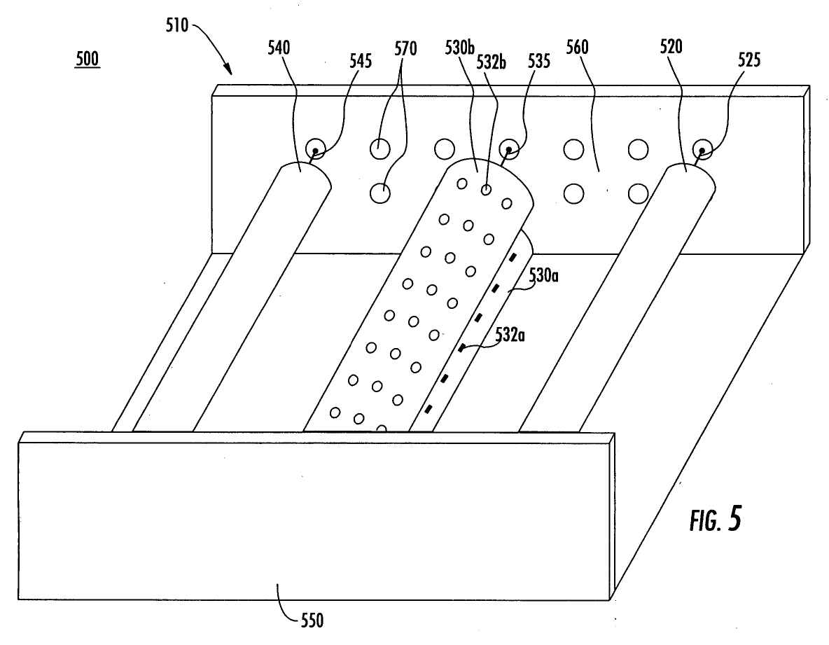

As shown in FIG. 5, the skin graft preparing device 500 can include a base structure 510, one or more skin feeding structures 520 rotatably coupled to the base structure 510. The skin graft preparing device 500 also includes one or more skin preparing structures 530a and 530b rotatably coupled to the base structure 510 for preparing the skin that is fed from the skin feeding structure 520. Further, one or more skin collecting structures 540 is rotatably mounted to the base structure 510 for collecting the prepared skin from the skin preparing structures 530a and 530b. The skin feeding structures 520, the skin preparing structures 530a and 530b, and the skin collecting structures 540 can be removably coupled to the base structure 510 for a variety of arrangements, including moving collecting skin to a skin grafting site on a patient.

The base structure 510 forms a mounting base that can be used for rotatatly coupling the skin feeding structures 520, the skin preparing structures 530a and 530b, and the skin collecting structures 540 of the skin graft preparing device 500. The base structure 510 can be constructed, of any rigid material suitable for supporting the components of the skin graft preparing device 500. For instance, the base structure 510 can be constructed of a high strength ceramic or metal such as surgical steel. Preferably, the base structure 510 can be constructed of a high strength material that can withstand medical industry standard sterilization processes without any degradation.

The base structure 510 can include one or more first mounting structures 550 and one more second mounting structures 560. Although the first mounting structure 550 and the second mounting structure 560 are shown as opposing wall-like structures of the base structure 510, the invention is not limited in this regard. For instance, the first mounting structures 550 and the second mounting structures 560 can be individual mounting structures that can support only one component of the skin grafting device 500. Thus, the first mounting structures 550 and the second mounting structures 560 can be individual square or cylindrical posts instead of unitary wall-like units. Further, it should be noted that the base structure 510 can include spaced post anchors for anchoring posts to the base structure 510 in a variety of spatial configurations.

Additionally, the first mounting structure 550 and the second mounting structure 560 can include a multitude of rotating elements 570 organized in pairs for rotatably coupling the skin feeding structures 520, the skin preparing structures 530a and 530b, and the skin collecting structures 540. The rotating elements 570 can include any suitable structure for rotatably coupling the components of the skin graft preparing device 500 to the base structure 510. In one example, the structure of the rotating elements 570 can include a recess that corresponds to the shape of a portion of the skin feeding structure 520, the skin preparing structures 530a and 530b, and the skin collecting structure 540, to ensure a secure coupling without the need for any moving parts. Nevertheless, the invention is not limited in this regard as the structure of the rotating element 570 can include movable portions such as a nut that can secure a portion of the skin feeding structure 520, the skin preparing structures 530a and 530b, and the skin collecting structure 540 to the rotating element 570.

Additionally, the rotating elements 570 can include an suitable mechanism for imparting rotational movement to the skin feeding structure 520, the skin preparing structures 530a and 530b, and the skin collecting structure 540. The suitable rotating mechanism can include an electric motor for imparting rotational motion. Nevertheless, the invention is not limited in this regard as the rotating mechanism of the rotating element 570 can include a hand crank to allow a user to control the rate and speed of rotation. Furthermore, it should be noted that the rotating mechanism can be included with only one pair of rotating elements 570 in-between which the skin feeding structure 520, the skin preparing structures 530a and 530b, and the skin collecting structure 540 can be mounted.

The rotating mechanism can be adapted to rotate the skin feeding structure 520, the skin preparing structures 530a and 530b, and the skin collecting structure 540 in both the clockwise and counterclockwise directions. Nevertheless, the invention is not limited to one direction of rotation as some pairs of rotational elements can be configured for imparting clockwise rotation while other are configured to impart counter clockwise rotation.

Additionally, it should be noted that the invention is not limited to an arrangement where each rotating element 570 includes a rotating mechanism. The rotational motion imparted by the rotation elements 570 can be controlled by a single rotating mechanism for the entire graft preparing device 500. For example, one rotating mechanism can be disposed within the first mounting structure 550 and connected to each rotating element 570. Such a rotating mechanism can impart rotating motion to each rotating element 570 at a uniform speed. Uniform rotational speed can ensure smooth operation of the skin graft preparing device 500.

The skin feeding structure 520, the skin preparing structures 530a and 530b, and the skin collecting structure 540 are all preferably cylindrical structures having a circular shape that is rotatably coupled to the base structure 510. They also can each respectively include an engagement portion 525, 535, and 545 that can be rotatably coupled to a pair of rotational elements 570. Once rotatably coupled, the rotation mechanism of rotational elements 570 can impart rotational motion to skin feeding structure 520, the skin preparing structures 530a and 530b, and the skin collecting structure 540, around an axis respectively defined by engagement portion 525, 535, and 545. The skin feeding structure 520, the skin preparing structures 530a and 530b, and the skin collecting structure 540 can be constructed of a material, such as surgical steel, that can undergo medical industry standard sterilization processes without degradation. Nevertheless, the invention is not limited in this regard as they can also be constructed of other materials and considered disposable.

Although the skin feeding structure 520, the skin preparing structures 530a and 530b, and the skin collecting structure 540 are each preferably a circular cylindrical body, the invention is not limited in this regard. They can include any suitable cylindrical body having a polygonal shape, such as a hexagonal shape. It should also be noted that they can each have a different shape and size from one another.

The skin preparing structures 530a and 530b can include two members that act in conjunction with one another to prepare skin. The skin preparing structure 530a can include a multitude of cutting elements 532a, such as raised sharp metal protrusions and pointed pin structures. The skin preparing structure 530b can include a multitude of holes or depressions 532b that are shaped and sized to correspond to the shape and size of the cutting elements 532a. The holes 532b and the cutting elements 532a are spatially arranged to synchronously match up with each other during rotation. In such an arrangement, each cutting element 532a can briefly protrude into the corresponding hole 532b during rotation to at least partially pierce the skin.

During operation, skin is rotated over and around the skin preparing structure 530. As the skin preparing structures 530a and 530b synchronously rotate, the cutting elements 532a are forced through the skin and into the holes 532b, thereby providing a multitude of small incisions in the skin. Cutting a portion of skin with multiple small incisions can be referred to as meshed skin which is capable of being expanded beyond original size without detrimentally tearing and destroying the cells of the skin.

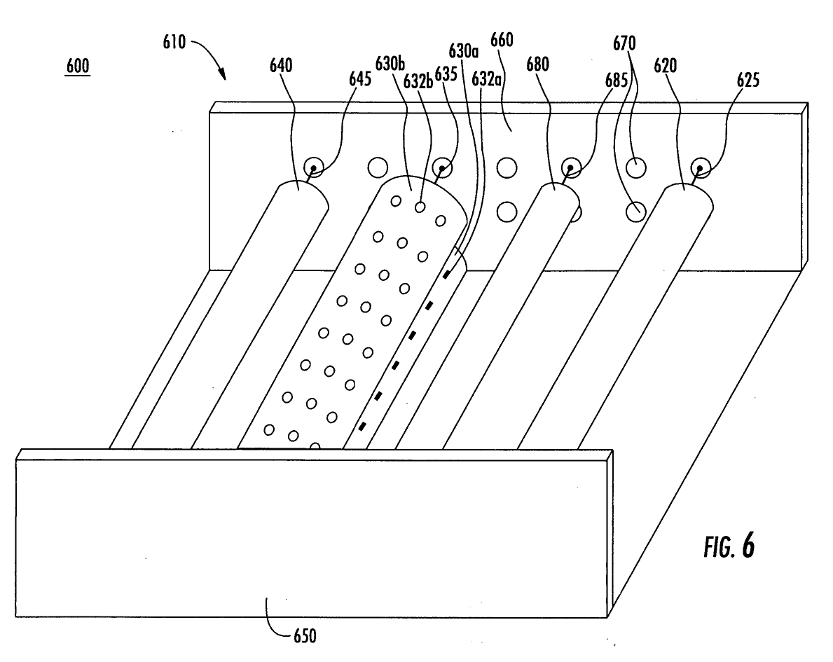

There is shown in FIG. 6, another embodiment of a skin graft preparing device in accordance with the inventive arrangements In this embodiment, a skin graft preparing device 600 can include a base structure 610, a skin feeding structure 620, and skin preparing structures 630a and 630b, a skin collecting structure 640, and a dressing feeding structure 680. The dressing feeding structure 680 can include any cylindrical body and can be rotatably coupled to the base structure 610 in-between a pair of rotating elements 670 and at engagement portion 685. The rotational mechanism of rotation element 670 can impart rotation movement to the dressing feeding structure 680 around an axis defined by engagement portion 685.

The dressing feeding structure 680 can be constructed of a material, such as surgical steel, that can undergo medical industry standard sterilization processes without degradation. Nevertheless, the invention is not limited in this regard as the dressing feeding structure 680 can be constructed of other materials and considered disposable. Additionally, the dressing feeding structure 680 can be removably coupled to the base structure 610 to allow the position of the dressing feeding structure 680 to be moved and for new dressing feeding structures 680 to replace depleted dressing feeding structures 680.

Referring to FIGS. 6 through 8, a skin graft preparing device 600 is shown in multiple arrangements. The skin graft preparing device 600 is shown with the skin feeding structure 620, and skin preparing structures 630a and 630b, a skin collecting structure 640, and a dressing feeding structure 680 rotatably coupled to the base structure 610 at various heights and lengths along the first mounting structure 650 and second mounting structure 660. Thus, the skin graft preparing device 600 is adjustable and configurable to a variety of arrangements.

In FIG. 6, the dressing feeding structure 680 is shown rotatably coupled to the base structure 610 between the skin feeding structure 620 and the skin preparing structures 630a and 630b. Such an arrangement allows for a dressing, such as a basal layer of collagen-glycosaminoglycan and/or other skin substitutes and dressings, to be coupled to the skin before the skin is prepared by the skin preparing structure 630. Thus, both the skin and the dressing can be prepared by the skin preparing structure.

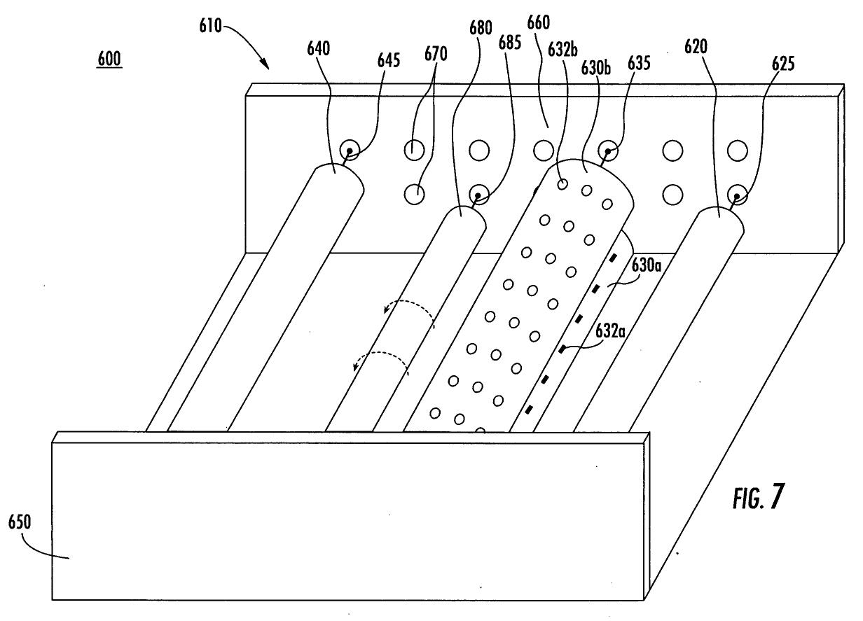

An alternative arrangement of skin graft preparing device 600 is shown in FIG. 7. Here, the dressing feeding structure 680 is shown rotatably coupled to the base structure 610 between the skin preparing structures 630a and 630b and the skin collecting structure 640. Such an arrangement allows a dressing to be coupled to the skin after the skin has been prepared by the skin preparing structures 630a 630b, therefore, the skin preparing structure only prepares the skin and does not manipulate the dressing.

FIG. 7 also illustrates that the dressing feeding structure 680 may be rotatably coupled to the base structure 610 with the dressing feeding structure 680 rotating in the counter-clockwise direction, as indicated by the dotted arrows. Such an arrangement with the dressing feeding structure 680 dispensing dressing in the counter-clockwise direction will couple the dressing to a bottom side of the skin. Thus, the skin with dressing coupled to the bottom side of the skin will be collected by the skin collecting structure 640.

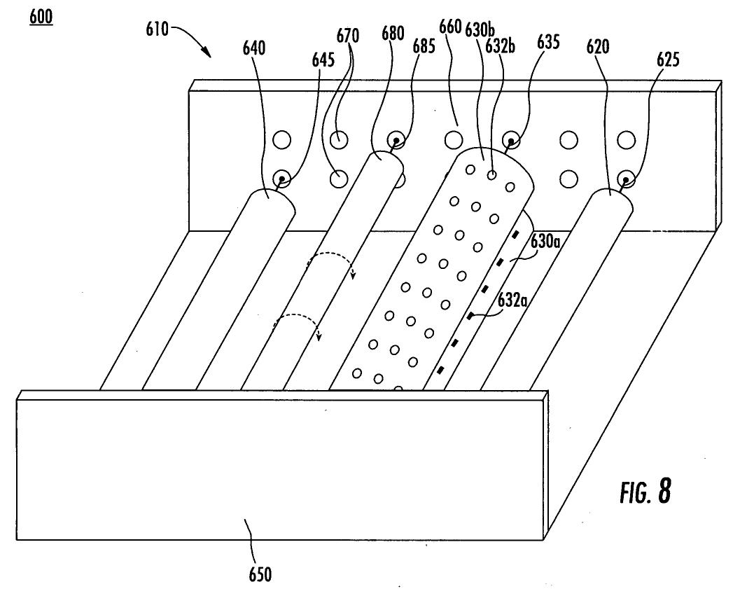

FIG. 8 shows an alternative arrangement to the arrangement shown in FIG. 7. FIG. 8 shows the dressing feeding structure 680 rotatably coupled to the base structure 610 with the dressing feeding structure 680 rotating in the clockwise direction, as indicated by the dotted arrows. Such an arrangement with the dressing feeding structure 680 dispensing dressing in the clockwise direction will couple the dressing to a top side of the skin. Thus, the skill with dressing coupled to the to side of the skin will be collected by the skin collecting structure 640.

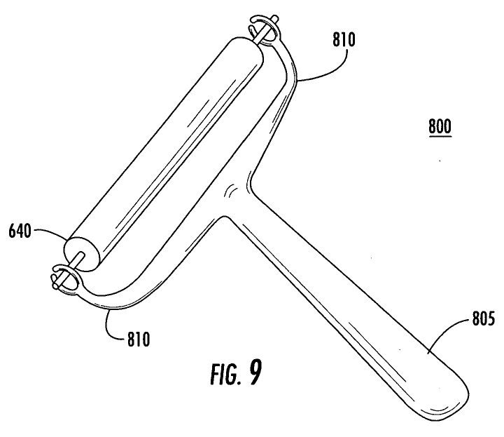

FIG. 9 illustrates one embodiment of a skin applying device. The skin applying device 800 can include and handle 805 and coupling arms 810 for rotatably and removably coupling the skin collecting structure (640 or 540) of the skin graft preparing device (500 or 600). Once rotatably coupled, the prepared skin can be rolled on the patient. Further, the skin applying device 800 can also be used to rotatably and removably couple the other rotating members within the surgical device 100, such as skin supporting structure 150, or the skin preparing devices 500 or 600. The skin applying device 800 also allows the skin collecting structure of the skin graft preparing device to be directly used in applying the skin to the patient. The skin applying device 800 can be constructed of any suitable material that can undergo medical industry standard sterilization process. Alternatively, the skin applying device 800 can be constructed of any other suitable material and can be considered disposable.

In one example of operation of skin graft preparing device 600 shown in FIG. 7, a portion of skin may be provided on skin feeding structure 620. The rotational mechanism of the rotational element 670, to which the skin feeding structure 620 is rotatably coupled, imparts rotational motion to the skin feeding structure 620. During rotation, skin can be fed to the skin preparing structures 630a and 630b. Skin can be wrapped around either skin preparing structure 630a or 630b and the cutting elements 632a will synchronously align with holes 632b to produce a multitude of incisions in the skin. Alternatively, the skin may be passed between skin preparing structures 630a and 630b. The skin can then be transferred to dressing feeding structure 680 which is rotating in the counter-clockwise direction. Thus, a layer of dressing will be coupled to the bottom side of skin. Finally, the skin with dressing coupled to the bottom side thereof can be collected by the skin collecting structure 640. Importantly, the skin collecting structure 640 will prevent the skin from bunching upon itself and possibly becoming entangled and/or damaged. The skin collecting structure 640 with prepared skin can be removed with the skin applying device 800 and then applied to skin by rolling the skin in onto the patient.

A skin graft preparing system is also provided in accordance with the inventive arrangements. The skin graft preparing system can include a surgical device such as surgical device 100 shown in FIG. 1. Additionally, the skin graft preparing system can include a skin graft preparing device such as the skin graft preparing device 500 shown in FIG. 5. Further, the skin graft preparing system can also include the skin applying device 800 shown in FIG. 9.

In the skin graft preparing system, the skin supporting structure 150 of the surgical device 100 can also be used in the skin graft preparing device 500. In such an arrangement, the surgical device 100 and the skin graft preparing device 500 can include comparable structure that allows the skin supporting structure 150 to be removably and rotatably coupled thereto. Such removable couplings allow the skin supporting structure 150 with collected skin to be detached from the surgical device 100 and coupled to the skin graft preparing device to be used as the skin feeding structure 520. Further, the skin applying device 800 can used to transfer the skin supporting structure 150 to the skin preparing device without any manipulation of the separated skin. Thus, only one structure is needed to operate as the skin supporting structure 150 and the skin feeding structure 520. Further, it should be noted that skin graft preparing system can include a skin graft preparing device 600 shown in FIG. 6 which includes a dressing feeding structure 680 rotatably and removably coupled to base structure 610.

In operation, skin is separated from a patient's body and collected on the skin supporting structure 150, which can be removably coupled to the surgical device 100. With the skin collected on the skin supporting structure 150 and using the skin applying device 800, the skin supporting structure 150 can be detached from the surgical device 100. The skin supporting structure 150, with the collected skin, can then be coupled to the skin graft preparing device 500 and used as the as the skin feeding structure 520. In this arrangement, skin is collected with surgical device 100 and transferred to skin graft preparing device 500 without any human manipulation that can introduce infection and possibly damage the separated skin. Once the skin is prepared, the skin collecting structure 640 can be removed with the skin applying device 800 and used for directly applying the skin to the patient. In such an arrangement, the prepared skin is collected and prepared without any human manipulation of the skin. In an alternative arrangement, the skin collected on the skin supporting structure 150 can be applied directly to the patient using the skin applying device 800 and without any skin preparation.

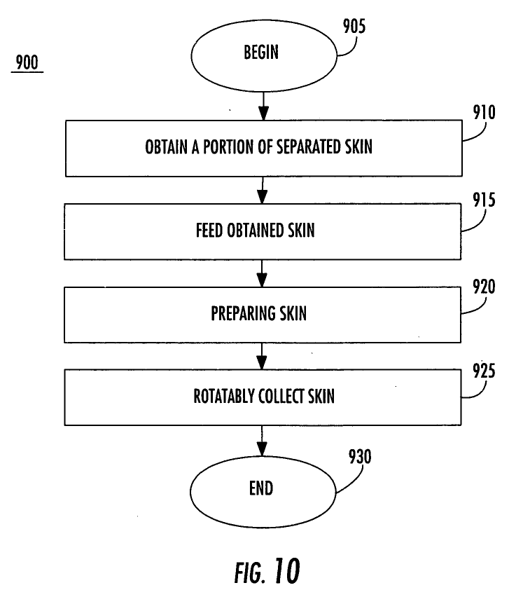

Also in accordance with the inventive arrangements, a method of preparing a skin graft is provided and shown in a flow chart in FIG. 10. The method 900 includes multiple steps that can generally be completed in any order. Additionally, while method 900 can be practiced with the skin graft preparing system discussed above, the method 900 is not limited in this regard and can be practiced with any suitable devices.

The method 900 can begin at step 905. In step 910, a portion of separated skin is obtained. While step 910 can usually include separating skin from a patient's body, the method is not limited in this regard. For example, the separated skin can also be obtained from another source, such as a from a supply of portions of separated skin. Furthermore, it should be noted that the term skin is not limited to skin harvested from a patient's body and can include synthetic skins and dressings.

In step 915, the skin can be fed so that it can be prepared. Feeding skin can involve continuously providing portions of the skin to a skin preparing device. For instance, skin can be fed by unrolling a portion of rolled skin and extending the unrolled portion so that it can be prepared. Alternatively, feeding skin can involve a one time action such as placing skin on a particular structure. For instance, an entire portion of separated skin that is to be prepared can be placed directly on a skin preparing structure, and therefore, the invention is not limited to continuously providing portions of skin.

In step 920, skin can be prepared. Skin can be prepared in a variety of manners that are within the scope of method 900 and can include preparing by cutting a multitude of incisions in the skin and/or by placing a dressing in contact with the skin. In one arrangement, the skin can be prepared by first cutting a plurality of incisions in the skin and then placing a dressing in contact with the skin. Such an arrangement can result in a skin combined with a dressing where only the skin has a multitude of incisions.

In an alternative arrangement, the skin can be prepared by first placing a dressing in contact with the skin and then cutting a multitude of incisions in the skin and/or the dressing. Thus, preparing step 920 can include cutting a multitude of incisions in the skin and dressing combination where only the skin is cut; however, preparing step 920 also includes cutting a multitude of incisions in the skin and dressing combination where both the skin and the dressing is cut. It should be noted that in preparing step 920, the dressing can be placed on top of the skin and on the bottom of the skin. Further, it should also be noted that the preparing step includes simply placing a dressing in contact with the skin without cutting incisions in either the skin or the dressing.

In step 925, the skin can be rotatably collected where the prepared skin can be progressively wrapped. In step 925, the prepared skin can be evenly distributed on a rotatable structure. In such an arrangement, the skin is not entangled upon itself and is immediately ready for patient use. The method 900 can end at step 930. Alternatively, any of the steps of method 900 can be repeated.

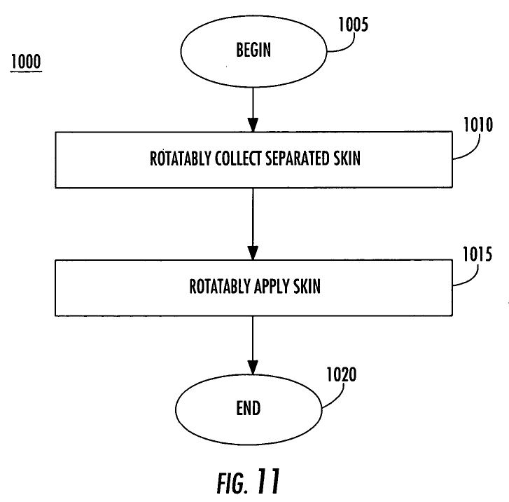

Also in accordance with the inventive arrangements, a method of obtaining and applying a skin graft is provided and shown in a flow chart in FIG. 11. The method 1000 includes multiple steps that can generally be completed in any order. Additionally, while method 1000 can be practiced with the skin surgical device discussed above, the method 1000 is not limited in this regard and can be practiced with any suitable devices.

In step 1005, method 1000 can begin. In step 1010, skin can be rotatably collected. Rotatably collecting skin includes wrapping skin around a roller while the skin is being separated from a patient's body; nevertheless, the invention is not limited in this regard as the skin can be rotatably collected after the skin is separated. Additionally, rotatably collecting skin can further include collecting the skin under tension so that the skin is evenly distributed on the roller.

In step 1015, the skin that has been rotatably collected can be rotatably applied to the patient's body. Thus, the same roller that is used to collect the skin can also be used to apply the skin. Rotatably applying the separated skin can include unwrapping skin from the roller while applying the skin to the patient's body. Skin is removed and unwrapped from the roller to apply the skin to the patient's body without any manipulation of the skin. Such a method of using a roller that has separated skin wrapped around it to directly apply the skin to the patient's body does not require any direct manipulation of the skin. Therefore, such a method limits the chance of infection due to human manipulation of the skin and can further eliminate the opportunities for human error.

While the preferred embodiments of the invention have been illustrated and described, it will be clear that the invention is not so limited. Numerous modifications, changes, variations, substitutions and equivalents will occur to those skilled in the art without departing from the spirit and scope of the present invention as described in the claims.

1. A surgical device for separating skin from a body, comprising:
a housing having a front end defining a forward direction and a rear end defining a rear direction, said housing for moving along skin in the forward direction; and
cutting structure moveably coupled to said housing for separating at least one portion of skin from the body, said cutting structure having a cutting portion;
wherein said cutting portion cyclically translates in the forward direction and the rear direction.

2. The surgical device according to claim 1, wherein said housing includes a skin contacting surface, said cutting portion translating substantially parallel to said skin contacting surface.

3. The surgical device according to claim 1, wherein said cutting portion translates a uniform forward distance;
whereby said cutting structure separates skin at a uniform depth.

4. The surgical device according to claim 1, wherein said housing includes a skin contacting surface, said cutting portion translating at an angle to said skin contacting surface.

5. The surgical device according to claim 4, wherein said cutting portion translates a uniform distance; whereby said cutting structure separates skin at a uniform depth.

6. The surgical device according to claim 1, further comprising a suction source for movably securing said housing to the skin.

7. The surgical device according to claim 6, wherein said suction source is coupled to at least one suction port located in said housing, said suction source creating a suction through said at least one suction port to movably secure said housing to the skin.

8. The surgical device according to claim 1, further comprising a skin supporting structure rotatably coupled to said housing;
whereby the skin supporting structure provides tension to and collects the separated skin.

9. The surgical device according to claim 8, wherein said skin supporting structure is removably coupled to said housing.

10. The surgical device according to claim 8, wherein said skin supporting structure comprises at least one attachment structure for attaching skin to said skin supporting structure.

11. The surgical device according to claim 8, wherein said skin supporting structure comprises a cylindrical body for receiving separated skin.

12. The surgical device according to claim 1, wherein said housing includes a guard to prevent unintentional skin separation.

13. A surgical device for separating skin from a body, comprising:
a housing;
a cutting structure movably coupled to the housing for separating at least one portion of skin from a body, said cutting structure having an elongated cutting edge defining a first axis, said cutting structure and said elongated cutting edge substantially contained within a first plane; and
wherein said elongated cutting edge cyclically translates in said first plane substantially perpendicular to said first axis.

14. The surgical device according to claim 13, further comprising a suction source for movably securing said housing to the skin.

15. The surgical device according to claim 14, wherein said suction source is coupled to at least one suction port located in said housing, said suction source creating a suction through said at least one suction port to movably secure said housing to the skin.

16. The surgical device according to claim 13, further comprising a suction source coupled to at least one suction port located in said housing, wherein said suction source creates a suction through said at least one suction port to movably secure said housing to the skin.

17. The surgical device according to claim 13, further comprising a skin supporting structure rotatably coupled to said housing;
whereby the skin supporting structure provides tension to and collects the separated skin.

18. The surgical device according to claim 17, wherein said skin supporting structure comprises at least one attachment structure for attaching skin to said skin supporting structure.

19. A skin graft preparing device, comprising:
a base structure;
at least one skin feeding structure coupled to said base structure for feeding skin;
at least one skin preparing structure coupled to said base structure for preparing the, skin fed from said skin feeding structure;
at least one skin collecting structure coupled to said base structure for collecting the prepared skin from said skin preparing structure.

20. The skin graft preparing device according to claim 19, wherein said base structure includes at least one first mounting structure and at least one second mounting structure for mounting said at least one skin feeding structure, said at least one skin preparing structure, and said at least one skin collecting structure.

21. The skin graft preparing device according to claim 20, wherein said first mounting structure and said second mounting structure include a plurality of rotating elements for rotatably coupling at least one skin feeding structure, at least one skin preparing structure, and at least one skin collecting structure.

22. The skin graft preparing device according to claim 21, wherein said rotating elements are spatially arranged along said first mounting structure and said second mounting structure to allow for variable spatial coupling of said at least one skin feeding structure, said at least one skin preparing structure, and said at least one skin collecting structure.

23. The skin graft preparing device according to claim 21, wherein said rotating elements impart rotational motion to at least one of said at least one skin feeding structure, said at least one skin preparing structure, and said at least one skin collecting structure.

24. The skin graft preparing device according to claim 19, wherein said at least one skin feeding structure, said at least one skin preparing structure, and said at least one skin collecting structure are removably coupled to said base structure.

25. The skin graft preparing device according to claim 19, further including a dressing feeding structure rotatably and removably coupled to said base structure.

26. The skin graft preparing device according to claim 25, wherein said dressing feeding structure is rotatably coupled to said base structure between said skin feeding structure and said skin preparing structure for coupling dressing to unprepared skin.

27. The skin graft preparing device according to claim 25, wherein said dressing feeding structure is rotatably coupled to said base structure between said skin preparing structure and said skin collecting structure for coupling dressing to prepared skin.

28. The skin graft preparing device according to claim 25, wherein said dressing feeding structure is rotatably coupled to said base structure, said dressing feeding structure rotating in the clockwise direction so that said dressing from dressing feeding structure couples to a top side of said skin.

29. The skin graft preparing device according to claim 25, wherein said dressing feeding structure is rotatably coupled to said base structure, said dressing feeding structure rotating in the counter-clockwise direction so that said dressing from dressing feeding structure couples to a bottom side of said skin.

30. A skin graft preparing system, comprising:
a surgical device having a housing with a front end defining a forward direction and a rear end defining a rear direction, said housing for moving along skin in the forward direction, a cutting structure moveably coupled to said housing for separating at least one portion of skin from the body, said cutting structure having a cutting portion, said cutting portion cyclically translating in the forward direction and the rear direction;
a skin graft preparing device having a base structure, at least one skin feeding structure rotatably and removably coupled to said base structure for feeding skin, at least one skin preparing structure rotatably coupled to said base structure for preparing the skin fed from said skin feeding structure, at least one skin collecting structure rotatably coupled to said base structure for collecting the prepared skin from said skin preparing structure; and
wherein said skin supporting structure from said surgical device provides said skin feeding structure of said skin graft preparing device.

31. The skin graft preparing system according to claim 30, wherein said skin graft preparing device further includes a dressing feeding structure rotatably and removably coupled to said base structure.

32. The skin graft preparing system according to claim 30, further comprising a skin applying device;
wherein the skin applying device rotatably couples to at least one of the skin supporting structure and the skin collecting structure.

33. A method of preparing a skin graft, comprising the steps of:
obtaining a portion of separated skin;
preparing the portion of separated skin; and
rotatably collecting the prepared portion of separated skin.

34. The method of preparing a skin graft according to claim 33, wherein said rotatably collecting step includes evenly distributing the prepared skin on a rotatable structure.

35. The method of preparing a skin graft according to claim 33, further comprising the step of feeding obtained separated skin for said preparing step.

36. The method of preparing a skin graft according to claim 33, wherein said preparing step includes at least one of cutting a plurality of incisions in the separated skin and placing a dressing in contact with the separated skin.

37. The method of preparing a skin graft according to claim 36, wherein said preparing step includes first cutting a plurality of incisions in the separated skin and then placing a dressing in contact with the separated skin.

38. The method of preparing a skin graft according to claim 36, wherein said preparing step includes first placing a dressing in contact with the separated skin and then cutting a plurality of incisions in at least one of the separated skin and the dressing.

39. The method of preparing a skin graft according to claim 36, wherein the dressing is placed on a top surface of said separated skin.

40. The method of preparing a skin graft according to claim 36, wherein the dressing is placed on a bottom surface of said separated skin.

41. A method of obtaining and applying a skin grafting, comprising the steps of:
rotatably collecting separated skin;
rotatably applying separated skin; and
wherein the skin is rotatably collected and rotatably applied using the same rotatable structure.
